(19) United States
(12) Patent Application Publication
(10) Pub. No.: US 2024/0042231 A1
(43) Pub. Date: Feb. 8, 2024

(54) SYSTEM TO SUPPORT TREATMENTS BASED ON THE EXPOSURE OF A USER OR A SURFACE TO SOLAR RADIATION

(71) Applicant: SIHEALTH PHOTONICS S.R.L., Livorno (IT)

(72) Inventors: Emilio SIMEONE, Livorno (IT); Marco MORELLI, Oxford (GB)

(21) Appl. No.: 18/266,840

(22) PCT Filed: Mar. 31, 2021
(86) PCT No.: PCT/IB2021/052686

§ 371 Date: Jun. 13, 2023

(30) Foreign Application Priority Data
Dec. 23, 2020 (IT) 102020000032039

(51) Int. Cl. A61N 5/06; G16H 30/40; G16H 10/60; A61L 2/24; G16H 40/67; G16H 20/40

(52) U.S. Cl. CPC A61N 5/0616; G16H 30/40; G16H 10/60; A61L 2/24; G16H 40/67; G16H 20/40; A61N 2005/0627; A61N 2005/0657

(57) ABSTRACT

A treatment support system based on the exposure of a user or surface to solar radiation, comprising a server (WS), a plurality of terminals and one or more satellite and/or meteorological atmospheric data sources interfaced with each other, and a processing unit configured to process treatment administration operational information based on satellite and/or meteorological atmospheric data (D3), geolocation data (D2), and user or treated surface specific data (D1).

### INVENTION SECTOR

The invention relates to a solar radiation treatment support system based on the detection of the dose of solar radiation received by an individual, possibly also considering the effect due to the use of a substance taken systemically or applied topically (e.g., cream or patch) which has photosensitising effects, if used, or a photo protective cream, if applied, or both simultaneously, if used. The system can also be applied to support the disinfection of surfaces by photodynamic antimicrobial treatment in the case of inanimate surfaces (e.g., flooring or fabrics, such as clothes, gloves and masks) coated with antimicrobial spray or paint and exposed to solar radiation.

The system can be effectively used as a support for a variety of treatments based on exposure to solar radiation, which include, but are not limited to the following: photodynamic treatment, photo rejuvenation treatment, neurofibroma treatment, treatment of local skin reactions, hardening and desensitisation of the skin by ultraviolet solar radiation, solar polymorphic dermatitis treatment, heliotherapy, climate therapy and balneotherapy.

In particular, the system can be used as a support for treatment by solar radiation of numerous skin diseases, including but not limited to: actinic keratosis, psoriasis, vitiligo, acne, eczema, atopic dermatitis, tumours and pre-tumours of the skin, DNA damage of the skin, jaundice, cutaneous lymphoma, lichen planus and circadian skin disorders. Moreover, the system can also be effectively applied for phototherapy not specifically concerning the skin, as in the case of ocular phototherapy.

The system of the invention may also find application in physiological treatments based on solar radiation (e.g., in connection with the production of vitamin D) related to photoageing effects, carried out with or without the use of supplements or stimulators of the production of the substance of interest (e.g., vitamin D) from solar radiation and with or without applied photoprotective solar filters.

More particularly, the system may comprise a digital platform connected to portable telecommunications devices, which can be accessed by both patients and medical personnel, for planning and managing a treatment based on exposure to solar radiation.

The system is able to provide a predicted and/or real-time measurement of the radiation actually received by the individual, also considering the effect of any creams applied.

The system may also optionally include a wearable device connected to the portable telecommunication device and equipped with sensors to determine position, posture, and orientation of the individual relative to the position of the sun, so as to further increase the detection accuracy of the dose of solar radiation on the concerned areas of the skin, and/or to identify the mode of exposure of the individual to the sun, and, specifically, exposure to full light, in shade or partial shade, or fully shielded from solar radiation by comparing a UV sensor and a light meter with solar radiation measured from satellite data.

### BACKGROUND ART

It is known that phototherapeutic treatments (phototherapy) are healing techniques based on the use of light, that is, on an individual's exposure to a luminous radiation for a certain time interval. In general, phototherapy is often used to treat dermatological disorders (actinic keratosis, acne, psoriasis, eczema, skin cancers such as squamous cell carcinoma and basal cell carcinoma), eye disorders, sleep disorders (circadian rhythm disturbances, insomnia), deficiency or maintenance of nutrients essential to the health of the person (e.g., vitamin D) and some psychiatric diseases (seasonal affective disorder).

The light source used can be either a lamp (for phototherapy indoors) or, alternatively, solar radiation (for phototherapy outdoors or in a sunroom).

Solar radiation-based phototherapy is usually less expensive (no lamp is necessary), is more appreciated by patients, as it allows treatment outdoors (or, in any case, in environments exposed to natural light, such as a sunroom), and allows a more homogeneous exposure of the individual since solar radiation is diffused from the atmosphere and with a perfectly collimated direct component due to the distance of the source (the solar source is at an “infinite” distance, an effect obviously impossible to achieve under an artificial source indoors).

To date, one of the main negative aspects of phototherapy based on solar radiation, however, is the difficult management of the treatment due to uncertainty in the administration of the optimal dose of solar radiation in the specific spectral region in order to achieve the desired therapeutic effect, and the difficulty in predicting the optimal time interval of exposure on the desired day, and consequently the possibility of scheduling the appointment for the therapeutic session with the patient in the presence of a doctor and for the patient to plan their availability for the session.

Accurate monitoring of the dose of solar radiation is in fact necessary to obtain the best therapeutic results with minimal adverse/side effects (such as the risk of erythema/sunburn), also ensuring the administration of the minimum effective dose for the treatment. On the other hand, the prediction of the time interval for planning treatment is an operational necessity for clinical staff, whose uncertainty today constitutes an important limit to the use of solar phototherapy in different treatment centres. The situation is even more critical if, as is the case of several phototherapy treatments with indoor lamps, the patient should self-administer therapy in an outdoor setting (e.g., his/her home garden), in an environment that therefore does not have qualified technical personnel (e.g., health physicist) with specific radiometric instruments (e.g., spectrometers).

A particular case of phototherapeutic treatments based on solar radiation for which accurate detection/prediction of the dose of solar radiation is very important are those that include the use of photosensitising substances. For example, daylight PhotoDynamic Therapy (dPDT) consists of applying to one or more skin lesions a photosensitising substance, which is activated during exposure to sunlight and leads to the removal of pathological cells from the lesions. DPDT is commonly used to treat actinic keratosis, superficial basal cell carcinoma (sBCC), Bowen's disorder, neurofibroma and also for antimicrobial therapies and local skin reaction (LSR) treatments. DPDT may also be used for dermatological treatments for aesthetic purposes, such as photo-rejuvenation or photo-retouching of photo-aged and/or sun-damaged skin (particularly UV).

Another example is phototherapy for psoriasis or vitiligo, called PUVASOL therapy and based on the administration of photosensitisers orally, or applied in solution to the skin (e.g., Psoralens such as 8-MethoxyPsoralen) followed by a session exposed to SOLAR GRAPE radiation, (e.g., early morning in summer for 15-30 minutes). Other types of solar treatment do not include the use of photosensitising substances, but only direct exposure to sunlight, such as heliotherapy (for example for psoriasis), treatment of acne, desensitisation of the skin from UV radiation (skin thickening), treatment of polymorphic sun dermatitis (PLE), climatotherapy (for example, for the treatment of atopic dermatitis) and treatments for the deficiency or maintenance of nutrients essential for the health of the person (e.g., vitamin D). Again, the detection and prediction of effective solar radiation for each specific treatment is very important. All phototherapeutic treatments with solar radiation may be associated with the possible use of solar filters (sunscreens) with different spectral characteristics (e.g., typically in the UVB and GRAPE spectral regions), i.e., with different transmission/reflection spectra that must be considered to calculate the solar radiation that actually reaches the skin in a spectral band that is effective for the treatment in question. In fact, these filters are normally used to reduce any risks related to treatment (for example, skin erythema, even in areas of the body not affected by the treatment), but can also interfere with the effectiveness of the therapeutic effect of solar radiation and, if used, of the photosensitising drug, because they also filter the spectral component of the effective therapeutic solar radiation necessary for treatment.

Sometimes the photosensitising drug itself may contain therein a sunscreen specifically designed not to spectrally interfere with the spectral band of therapeutic interest, making the drug as a whole intrinsically safer to avoid adverse/side effects while ensuring maximum therapeutic efficacy.

By way of example, we report below typical configurations for some solar phototherapies showing how both a photosensitising drug and a solar filter may or may not be included:
dynamic solar phototherapy (dPDT) for the treatment of actinic keratosis carried out with or without applied sunscreen and with a photosensitising drug containing protoporphyrin IX (PpIX) applied to the skin lesion;
heliotherapy for the treatment of psoriasis (but also neurofibroma and Bowen's disorder) carried out with or without applied sunscreen and without any photosensitising drugs;
topical treatment of actinic keratosis by sun exposure with application on the skin lesion of a photo-active drug with integrated sunscreen, as in the case of the product “Eryfotona” by ISDIN (Cantabria Labs, Spain);
climatotherapy/balneotherapy for the treatment of atopic dermatitis carried out with or without applied sunscreen and with the skin lesion first immersed in sea water (in particular with high salt content, as in the case of the Dead Sea).

More generally, in the present invention the term “treatment” is understood also in the absence of a topical or systemic substance taken by the user and may relate to preventive and therapeutic effects for the benefit of the physiological systems of the person in the broadest sense, for example skin, eyes, immune system, circadian system, deficiency or maintenance of nutrients essential for the health of the person (e.g., vitamin D).

By way of example, publications WO2007106856, WO1999056827, WO1983002233 and WO2013036558, describe devices and methods for managing the phototherapy of an individual in a controlled manner with an artificial light source (e.g., with LEDs). However, these systems do not allow the management of a treatment with solar radiation, i.e., in the absence of a dedicated lamp or an illuminator that is part of the system.

Currently, there are also known systems that allow the detection of an individual's exposure to solar radiation according to the characteristics necessary for the different types of phototherapy with solar radiation.

As an example, WO2017153832 describes an individual's solar radiation exposure detection system consisting of a hand-held telecommunication device and wearable device.

However, this system does not allow the detection of effective solar radiation for a given solar phototherapy, nor to simultaneously consider the effect of a photosensitising drug, if applied, and/or of a solar filter, if applied, or if integrated into the photosensitising drug.

Furthermore, none of these methods include support for the post-therapy phase with processing photographs of the individual's skin lesions (e.g., via Artificial Intelligence). Currently, the issue of administering the correct dose of solar radiation is addressed with radiation monitoring mainly by in-situ radiometers and spectra. They can be used to monitor the dose of effective solar radiation in the different spectral regions of interest, considering, from time to time, the photobiological spectrum of action of interest for the specific treatment and for adverse/side effects (such as erythema/burning).

However, this is not generally the case in the medical field. In fact, natural solar radiation is not typically detected in the different spectral regions during phototherapy, apart from in some particular cases and in specially equipped centres of excellence with highly specialised physical-medical personnel. This is due to the high costs of purchasing and maintaining radiometers and spectrometers with a good level of accuracy over multiple spectral bands (as ideally required for phototherapy), the complexity of radiometric devices and their calibration, as well as the complex data processing procedures then needed to calculate effective solar radiation for each photobiological effect of interest.

In addition, accurate monitoring of solar radiation for phototherapy would simultaneously require more than one specific personal radiometer or dosimeter in order to control the dose relative to the multiple biological effects of solar radiation, both positive therapeutic effects (such as radiation effective for treatment) and adverse ones (such as erythema/sunburn). These devices should be manually set to thresholds defined based on the specific sensitivity of the individual to solar radiation (e.g., minimum erythemal dose—Minimal Erythemal Dose, MED—related to the sensitivity of the skin to UV radiation) and the spectral transmissivity of the solar filter that may be applied.

All this is naturally impracticable and, therefore, at present, solar phototherapy sessions are simply monitored and planned by simulating the expected solar radiation (often without even considering the different spectral bands), generally based on online weather forecasts and “typical” solar radiation climatic data in each geographical area for each period of the year, evidently with very limited accuracy and reliability.

This causes a lot of uncertainty in being able to ensure an adequate quality standard and to adequately evaluate the actual results of a solar phototherapy, also for a possible customisation and improvement of the therapeutic protocols, since solar radiation is the determining active factor but unfortunately it is not controlled reliably. In fact, solar phototherapy is currently difficult to carry out without direct and continuous control by clinical staff, so that the weather conditions and movements of the individual can be observed directly throughout the treatment.

Furthermore, the unavailability of accurate predictions of effective solar radiation for the treatment and its adverse/side effects makes the adoption of this type of treatment in clinical practices even more critical, despite all its potential benefits. In particular, to date there are no easily accessible and reliable systems for monitoring and predicting (time horizon of at least a few days) solar radiation weighted according to the therapeutic spectrum of action that, at the same time, also take into account the possible adverse/side effects of other spectral components of radiation, such as for example UV radiation.

In summary, the solutions currently in place do not allow accurate planning and monitoring of effective solar radiation for phototherapy considering all the spectra of action involved simultaneously (both positive, i.e., useful for treatment, and negative, i.e., related to side/adverse effects), including also the impact of potential photosensitising drugs and possible applied sunscreens (either in addition to or directly within the photosensitising drug).

These drawbacks also prevent the treatment of solar phototherapy from being administered in tele-dermatology, i.e., outside any hospital/clinical area (e.g., a patient's home).

Therefore, there is a need for a solar exposure measurement and control system of an individual or surface, capable of discriminating in detail the levels of sun exposure based on one or more parameters, such as the geographical location and, in the case of an individual, the specific phototherapy protocol of interest, the personal characteristics of the individual (such as UV sensitivity of the skin), any photosensitising drug, if applied, and the solar filter, if applied, so as to effectively support phototherapy. Similarly, in the case of inanimate surfaces subject to solar radiation, there is a need for a solar exposure measurement and control system for the purpose of photodynamic antimicrobial treatment of surfaces (e.g., flooring, clothing) for their disinfection based on the specific characteristics of the surface and the antimicrobial spray or paint applied on it to obtain disinfection from viruses and bacteria.

There is also a need for a digital solution that can effectively support both clinical staff and patients in the planning and conduct of phototherapy, also making it possible for the individual to do independently (for example, in the garden or in the sunroom of his/her home, or even during free outdoor activities), having however the treatment supervised remotely by the clinical staff in a reliable manner.

The same digital solution can also be effectively applied in a manner entirely similar to photodynamic treatments of inanimate surfaces (e.g., flooring, fabrics) for their disinfection, if coated with antimicrobial spray or paint and illuminated by solar radiation.

### OBJECT OF THE INVENTION

The present invention is intended to overcome the drawbacks of the already known solutions and to propose a system that allows both the planning and optimal and safe management of any phototherapy or treatment with solar radiation, maximising the amount of the spectral component of solar radiation effective for therapy or treatment, and minimising exposure to the potentially harmful spectral component (e.g., for health), while ensuring adherence to the treatment protocol, if any (e.g., in the case of clinical phototherapy).

In particular, the invention aims to provide a tool to optimise the simultaneous balance between the beneficial effect of exposure to solar radiation (e.g., preventive for the appropriate production of vitamin D, therapeutic or disinfectant) and related risks (e.g., erythema/burning, photoageing, cancer risk, or surface deterioration), and to recommend the best benefit/risk ratio both in terms of forecast and in terms of real-time monitoring, effectively and simultaneously taking into account the various factors involved, such as the impact of the therapeutic/beneficial solar radiation component, the impact of the solar radiation that could create adverse/undesirable effects, for example, the impact of photosensitising drugs (if applied, also considering the amount applied), or antimicrobial treatments for surfaces and sunscreens (if applied and/or if included in the photosensitising drug, also considering the amount applied).

### SUMMARY OF THE INVENTION

These objects have been achieved by providing a system and a method according to at least one of the attached claims.

A first advantage is that by means of the system of the invention the prediction of the solar radiation incident on the individual or on the surface effective for the treatment is able to identify the optimal time intervals of the day for the sun exposure session, to allow the maximum therapeutic/beneficial efficacy and the minimum risk of adverse/side effects, taking into account all the parameters involved, including the characteristics of the drug (if applied), or of the antimicrobial paint in the case of a surface (if applied), the characteristics of the photoprotective cream possibly applied, and the characteristics of the treated user (e.g., phototype, Minimum Erythema Dose—MED, concentration of vitamin D in the blood, etc.).

In addition, the system of the invention allows simultaneous real-time measurement of radiation doses effective for treatment and those potentially harmful in terms of adverse/side effects, in order to ensure the administration of the treatment with maximum benefit and minimum risk to the patient or the surface, taking into account the specific aforementioned characteristics of any drug, any photoprotective cream and the user, while ensuring maximum adherence to a therapeutic protocol, if any.

A third advantage is that by means of the system of the invention the measurement and prediction of the solar radiation incident on the individual effective for treatment are calculated by processing satellite and/or meteorological data relating to the state of the atmosphere, and, therefore, without the need for the use of optical sensors on the ground or wearable.

A further advantage is that it is possible to schedule and manage the solar treatment for both the individual and the medical staff remotely, with the individual not needing any dedicated lamp or device other than the portable telecommunication device, in order to have an effective treatment benefiting from the solar radiation in a controlled manner.

Such solar radiation measurement for effective treatment may also be made more accurate when a wearable posture and orientation sensor is integrated into the system, which makes it possible to measure the position of the individual's body parts with respect to the direction of the sun (azimuth and inclination with respect to zenith), performing a real-time three-dimensional measurement of solar radiation and its effective cumulative dose for treatment on each of the areas of the target body of the treatment.

### LIST OF DRAWINGS

These and additional advantages will be better understood by every person skilled in the art from the following description and the attached drawings, in which:

FIG. 1 shows the spectrum of action of photo-activation by solar radiation of a photosensitising drug containing protoporphyrin IX (PpIX).

FIG. 2 shows the spectrum of action of skin erythema during a user's exposure to solar radiation;

FIG. 3 shows a single graph showing both the action spectra of FIGS. 1 and 2;

FIG. 4 shows the spectral transmittance of a photoprotective cream with SPF50 and UVA protection;

FIGS. 5a and 5b show the graphs of the result of the spectral convolutions of the transmittance of the SPF50 photoprotective cream with the erythema spectrum of action and with the PpIX spectrum of action, respectively;

FIGS. 6a, 6b and 6c show the daily erythema solar irradiance on three different days of the year, expressed in terms of UV Index.

FIG. 7 schematically shows a digital system according to the invention.

FIG. 8 shows the photo-activation spectrum of riboflavin, a photosensitiser that can be used in the photodynamic antimicrobial treatment of surfaces;

FIG. 9 shows the spectrum of action of the skin vitamin D synthesis;

FIG. 10 shows in a single graph both the spectra of action of FIGS. 9 and 2 as an example of the beneficial and harmful effect of solar radiation, which can be balanced by the support of the proposed system even if no cream is applied or drug taken;

FIGS. 11a, 11b and 11c show the effective daily sunlight for vitamin D synthesis on three different days of the year, expressed in W/m².

### DETAILED DESCRIPTION

The accompanying drawings describe the preferred embodiments of a digital system according to the invention, supporting a treatment based on the exposure of an individual/user or surface to solar radiation.

In operation, the system leverages the measurement and prediction of effective solar radiation for treatment reaching the individual or surface (e.g., skin surface(s) of interest) derived from satellite and/or meteorological atmospheric data, including data relating to incident solar radiation.

Data relating to incident solar radiation means data permitting the measurement in real time or in anticipation of incident solar radiation.

By way of example, atmospheric data may include satellite Earth observation images and weather forecast data relating to environmental parameters in the geographical location occupied by such users or surfaces, and the numerical forecasts of atmospheric components, such as gases and particulates.

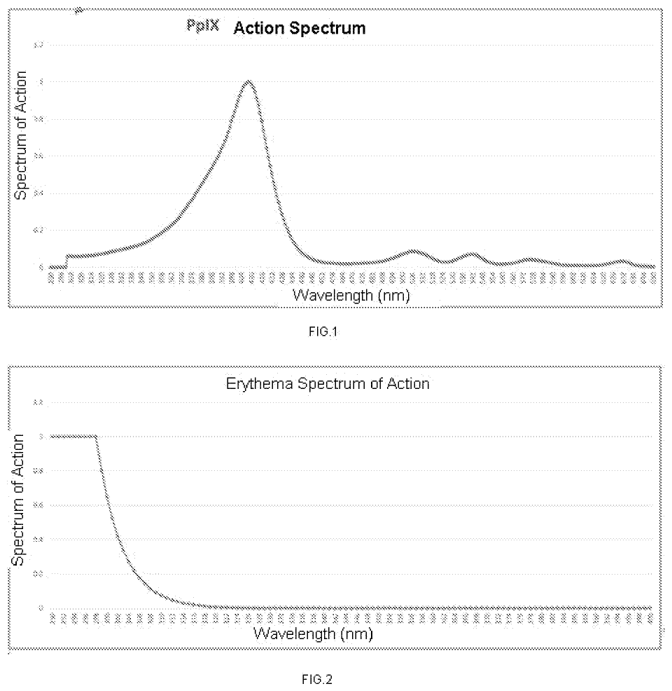

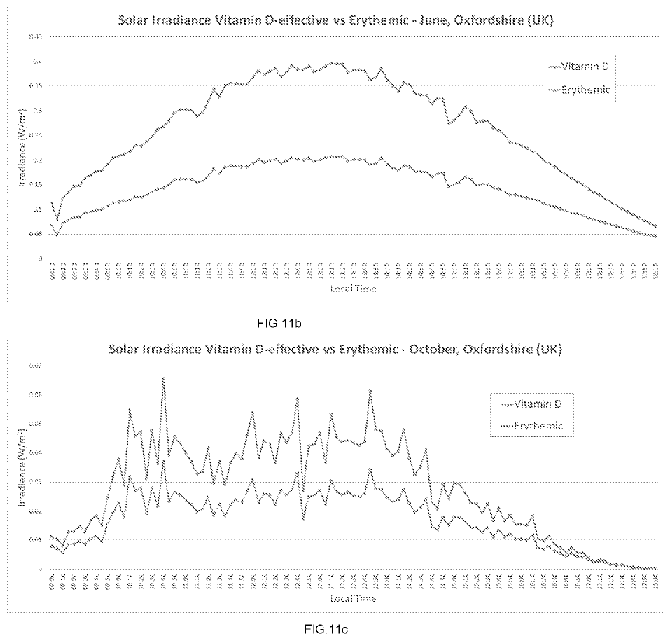

In particular, with reference to FIGS. 1-11, when applied to an individual/user, the system of the invention simultaneously takes into account:
the spectrum of photobiological action of specific phototherapy or the beneficial effect of solar radiation (e.g., vitamin D synthesis);
any other photobiological effects of interest related to treatment administration (e.g., erythema due to solar UV radiation or photoageing);
if applied, the spectral transmittance properties and their phototherapeutic spectrum of action of the specific photosensitising drug administered for treatment, also depending on the quantity per unit of surface applied (and determining its thickness);
if applied, the spectral transmittance of the solar filter (either separately or in combination with the photosensitising drug), also depending on the amount per unit of surface applied (and determining its thickness), considering both the interference with radiation effective for treatment and/or with the photosensitising drug (if applied) and the photoprotective effects for the individual.

Therefore, the operation of the proposed system amounts to the implementation of different personal dosimeters/radiometers associated with effective solar radiation both for the beneficial therapeutic effects and for the side effects and/or adverse effects related to the exposure to sunlight of the individual, and which take into account the filtering power of any sunscreens used.

The operation of the proposed system is preferably based on the analysis of Earth observation satellite images in combination with numerical weather forecasts and atmospheric modelling. It can be applied to any geographical location (e.g., with a typical spatial resolution between 1 km and 12 km), both in real time (e.g., with a time resolution of 1 minute) and to predict solar radiation in the future, obviously with accuracy and resolution that decrease proportionally to the time horizon of the forecast.

From a structural point of view, the proposed system consists of a multi-directional digital platform that integrates this method of prediction/monitoring of solar radiation based on atmospheric data (e.g., satellites) with the functions of treatment planning, treatment monitoring, remote supervision by clinical personnel, support for the post-therapeutic phase through image/photo processing of the area of interest and multi-user economic digital platform aimed at supporting solar phototherapy treatments.

In particular, in a preferred embodiment, the system operates in support of the design and operational management of solar phototherapies with photoprotective creams created to maximise the efficacy of treatment (e.g., creams with a particularly favourable transmission spectrum in the spectral region of interest for the therapy). Some examples of application in the case of solar photodynamic treatment (daylight PDT) for the treatment of actinic keratosis are given below in order to better clarify the innovative aspects and usefulness of the system of the invention.

In this case, the treatment is based on a photosensitising drug containing protoporphyrin IX (PpIX), which is photo-activated by solar radiation according to the spectrum of action (absorption) shown in FIG. 1.

This photo-activation serves to treat the skin lesion (actinic keratosis), typically requiring an effective solar radiation dose (i.e., weighted according to the PpIX spectrum of action) which is incident on the lesion of not less than 8 J/cm² for each session.

When a solar phototherapy session is completed, one of the main adverse effects to be taken care of is skin erythema (“redness”, which can also lead to burning).

This effect has the spectrum of action reported in FIG. 2 (ISO/CIE 17166:2019). The dose of erythema solar radiation (i.e., weighted according to the erythema spectrum of action) needed to experience an erythema (called Minimum Erythemal Dose—MED) is typically 300 J/m² for a person with medium light skin (phototype 2-3). In phototherapeutic treatments, 70% of the MED is normally considered a “risk threshold”; therefore, in this example we can consider the risk of erythema starting from a dose of solar erythema radiation of 210 J/m².

FIG. 3 shows a single graph of both the spectra of action mentioned (PpIX and erythema) to emphasise the difference and to visualise the distance (in spectral terms) between their peaks, which are in the UVB region in the case of erythema and in the UVA-visible region for PpIX.

When performing solar phototherapy, in some cases a photoprotective cream (sunscreen) is also used to avoid the risk of erythema in the individual.

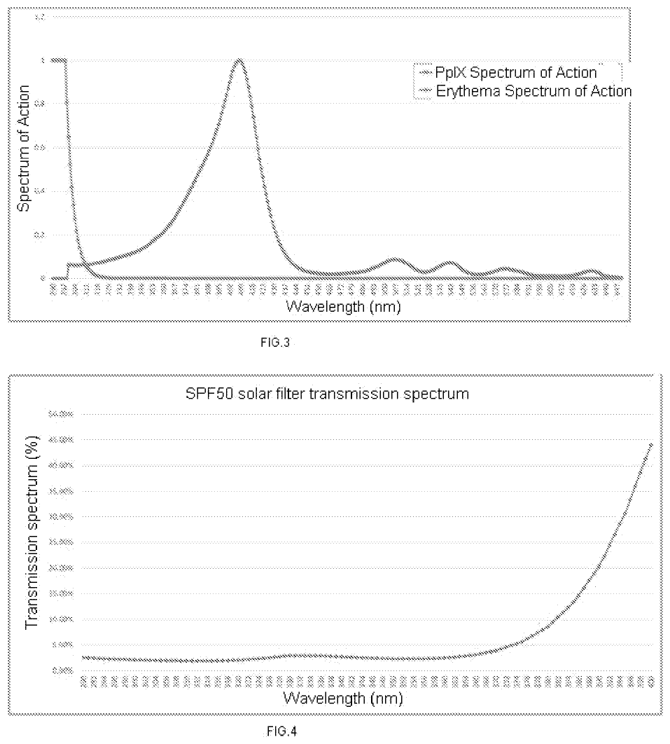

For example, in the graph of FIG. 4, we detail the spectral transmittance of a photoprotective cream with SPF50 and UVA protection (the one most commonly used for phototherapy, when used), assuming that it is applied in the standard amount of per unit surface of 2 mg/cm² on all skin exposed to sun radiation (including the lesion). The application of the sunscreen has a significant impact both in terms of protection from erythema but also as a “block” of the therapeutic action, that is, in this case of photo-activation of PpIX.

It is therefore necessary to appropriately assess both these impacts simultaneously, in order to effectively monitor and manage phototherapy, as the system proposed here makes possible.

In particular, the impact of the solar filter on both the erythema spectrum of action and the PpIX spectrum of action can be calculated by making a spectral convolution with the transmittance of the SPF50 photoprotective cream:

∫S_{SPF50}(λ)·S_{ery}(λ)dλ

∫S_{SPF50}(λ)·S_{PpIX}(λ)dλ

where S_{SPF50}(λ) is the spectral transmittance of the SPF50 photoprotective cream, S_{ery}(λ) is the erythema spectrum of action and S_{PpIX}(λ) is the spectrum of action (absorption) of PpIX.

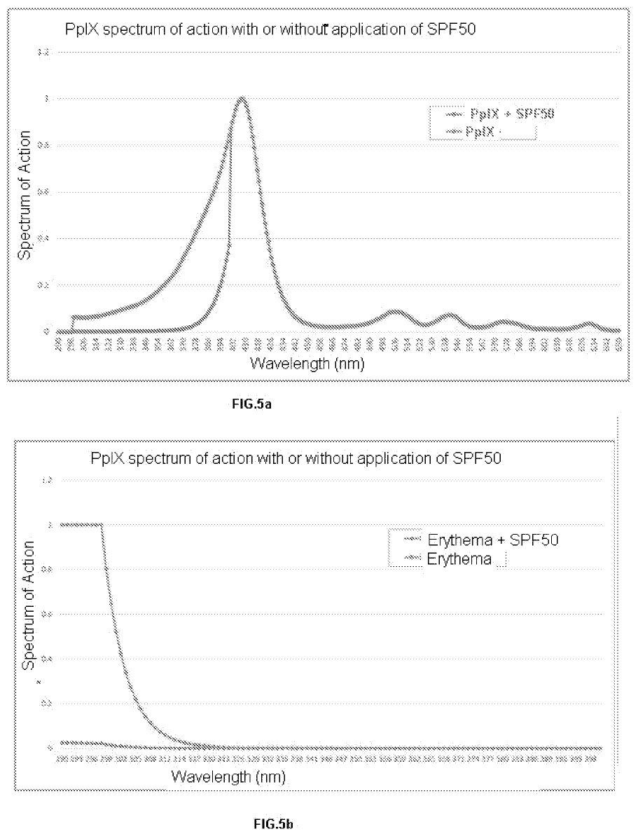

The result of these two convolutions is shown in the graphs of FIGS. 5a, 5b, which clearly show the significant impact of the photoprotective cream both in terms of reducing the therapeutic effect of PpIX (in particular in the UVA region) and in terms of protecting the erythema effect.

We now consider possible application scenarios in real conditions, i.e., on a sunny spring, summer or autumn day.

For this purpose, we will consider spectral solar radiation incident in England (Oxfordshire) to assess how these considerations and, therefore, the proposed innovative method play a fundamental role for the effective safe management of solar phototherapy, in particular highlighting the impact on the time needed to make the therapy efficacious, while also avoiding sunburn, with or without the application of photoprotective cream.

The effective solar radiation (erythema and PpIX) that reaches the skin of the individual when a photoprotective cream is not applied can be calculated as follows:

E_{ery}=∫E_{λ}(λ)·S_{ery}(λ)dλ

E_{PpIX}=∫E_{λ}(λ)·S_{PpIX}(λ)dλ

where E_{λ}(λ) is the spectral solar irradiance (typically measured in W/m²/nm), E_{ery} is the erythema solar irradiance (typically measured in W/m²), E_{PpIX} is the solar irradiance weighted according to the spectrum of action (absorption) of PpIX (typically measured in W/m²).

On the contrary, if a SPF50 photoprotective cream is applied, the formulas are slightly modified as follows:

E_{ery}=∫E_{λ}(λ)·S_{ery}(λ)·S_{SPF50}(λ)dλ

E_{PpIX}=∫E_{λ}(λ)·S_{PpIX}(λ)·S_{SPF50}(λ)dλ

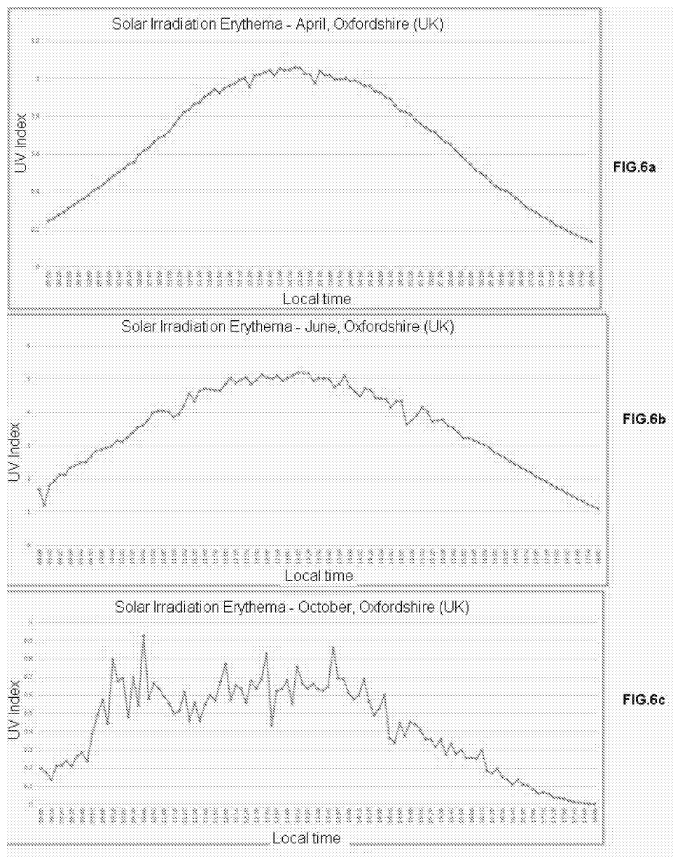

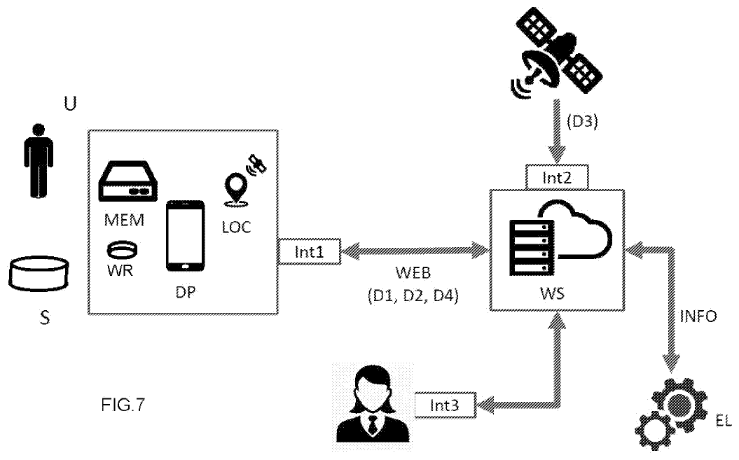

We now consider three typical non-rainy days in the Oxford region (UK) in April (spring), June (summer) and October (autumn), which could be selected for solar phototherapy (i.e., meeting dPDT standards in UK). The relative daily erythema irradiance expressed in terms of UV Index (UV Index) are given as an indication in the graphs of FIGS. 6a, 6b and 6c.

Considering the spectral solar radiation measured on the three days and that a dPDT session must last at least 2 hours according to the current European dermatological consensus for the daylight PDT protocol (Morton et al, J Eur Acad Dermatol Venereol. 2015 September; 29(9)), in the table below we provide the efficacy in terms of phototherapy (i.e., whether the threshold of 8 J/cm² of accumulated effective dose PpIX is exceeded or not), and in terms of protection from erythema for the sample individual considered (i.e., whether the threshold of 210 J/m² of accumulated erythema dose is exceeded or not) in three cases, i.e., for a 2-hour sun exposure session starting at 09:00 am, 12:00 pm and 4:00 pm with or without photoprotective cream (SPF50 sunscreen) applied.

| | April | June | October |
|---|---|---|---|
| 09:00 am | PpIX dose without | PpIX dose without | Dose PpIX without |
| to 11:00 | SPF50 filter = 8.01 | SPF50 filter = 25.08 | filter SPF50 = |
| am | J/cm² | J/cm² | 8.02 J/cm² |
| session | Erythema dose | Erythema dose | Erythema dose |
| (morning) | without SPF50 filter = | without SPF50 filter = | without SPF50 filter = |
| | 132.43 J/m² | 800.29 J/m² | 129.19 J/m² |
| | PpIX dose with | PpIX dose with | PpIX dose with |
| | SPF50 filter = | SPF50 filter = | SPF50 filter = |
| | 6.07 J/cm² | 18.14 J/cm² | 6.13 J/cm² |
| | Erythema dose with | Erythema dose with | Erythema dose with |
| | SPF50 filter = 3.67 | SPF50 filter = 19.56 | SPF50 filter = 3.58 |
| | J/m² | J/m² | J/m² |
| 12:00 pm | PpIX dose without | PpIX dose without | PpIX dose without |
| to 2:00 | SPF50 filter = 23.78 | SPF50 filter = 34.79 | SPF50 filter = 16.39 |
| pm | J/cm² | J/cm² | J/cm² |
| (mid-day) | Erythema dose | Erythema dose | Erythema dose |
| session | without SPF50 filter = | without SPF50 filter = | without SPF50 filter = |
| | 292.04 J/m² | 1441.78 J/m² | 189.77 J/m² |
| | PpIX dose with | PpIX dose with | PpIX dose with |
| | SPF50 filter = | SPF50 filter = | SPF50 filter = |
| | 18.29 J/cm² | 25.06 J/cm² | 12.71 J/cm² |
| | Erythema dose with | Erythema dose with | Erythema dose with |
| | SPF50 filter = 8.74 | SPF50 filter = 33.84 | SPF50 filter = 5.76 |
| | J/m² | J/m² | J/m² |
| 4:00 pm | PpIX dose without | PpIX dose without | Dose PpIX without |
| to 6:00 | SPF50 filter = 11.33 | SPF50 filter = 22.79 | filter SPF50 = |
| pm | J/cm² | J/cm² | 3.75 J/cm² |
| (evening) | Erythema dose | Erythema dose | Erythema dose |
| session | without SPF50 filter = | without SPF50 filter = | without SPF50 filter = |
| | 93.53 J/m² | 617.38 J/m² | 30.41 J/m² |
| | PpIX dose with | PpIX dose with | PpIX dose with |
| | SPF50 filter = | SPF50 filter = | SPF50 filter = |
| | 8.74 J/cm² | 16.52 J/cm² | 2.93 J/cm² |
| | Erythema dose with | Erythema dose with | Erythema dose with |
| | SPF50 filter = 3.26 | SPF50 filter = 15.57 | SPF50 filter = 1.05 |
| | J/m² | J/m² | J/m² |

The result of this simulation clearly demonstrates the usefulness of the proposed system in enabling the reliable and safe delivery of solar phototherapy sessions in accordance with the applicable therapeutic protocol.

In fact, we can see that
a) in April:
1. the morning session is therapeutically effective without the SPF50 filter, while it is not effective with the use of the SPF50 filter, and does not involve a risk of erythema for the individual under any circumstances; therefore, in this case, the use of the filter interferes with the effectiveness of the therapy and would be uselessly used to prevent erythema.
2. the mid-day session can be effective, but with risk of erythema if carried out without SPF50 filter (therefore, this possibility should be discarded), but, instead, it can safely and effectively take place if an SPF50 filter is applied;
3. the afternoon session can take place effectively and without risk of erythema both with and without SPF50 filter applied.
b) in June:
   all three sessions can only take place effectively if an SPF50 filter is applied, otherwise there is a risk of erythema for the individual.
c) In October:
1. the morning session is therapeutically effective without the SPF50 filter, while it is not effective with the use of the SPF50 filter, and does not involve a risk of erythema for the individual under any circumstances; therefore, again, the use of the filter interferes with the effectiveness of therapy and would be uselessly used to prevent erythema.
2. the mid-day session can take place effectively and safely with or without the SPF50 filter applied;
3. the afternoon session can never reach therapeutic efficacy.

Similar considerations and calculations can be made for any phototherapy with solar radiation, with or without any phototherapy product that may be applied and with or without any solar filter that may be applied.

What is shown with this example demonstrates the extreme usefulness of the proposed system. In fact, all key decisions regarding a solar phototherapy session, such as the time of its development, its actual duration, the date/season and whether or not to apply a photoprotective cream, depend heavily on spectral solar radiation and the simultaneous evaluation of the therapeutic effects (such as PpIX in the case of dPDT for actinic keratosis), and adverse/opposite effects (such as the risk of erythema). And these are decisions that have a strong impact on the risks and effectiveness of the treatment, as demonstrated.

Another preferred example embodiment of the system relates to support for balancing vitamin D synthesis against the risk of skin erythema in a user exposed to solar radiation without having applied any cream or taken any substance. The formulas and calculations provided above for the case without cream are also applied in a completely similar way, with the only difference that the spectrum of action to be considered for the beneficial effect is, in this case, that of vitamin D (FIG. 9); therefore, we will have:

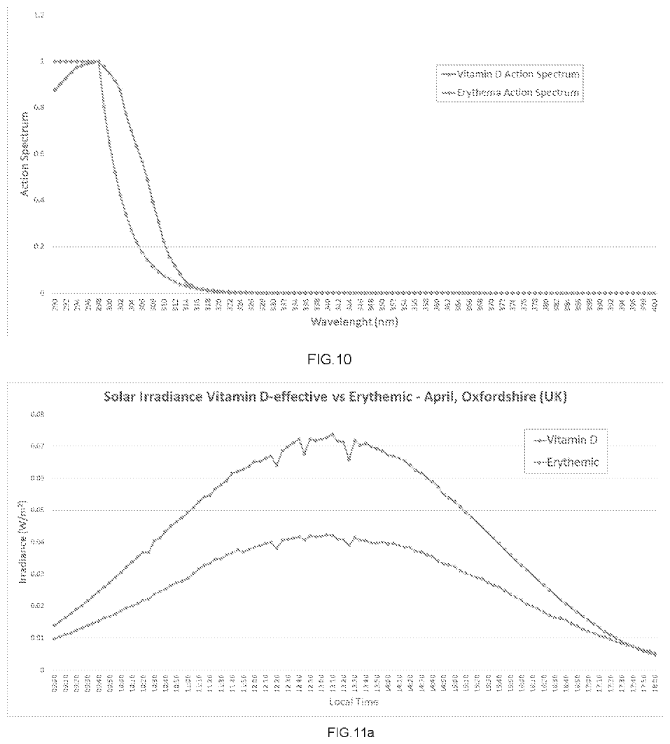

E_{vitD}=∫E_{λ}(λ)·S_{vitD}(λ)dλ

where E_{λ}(λ) is the spectral solar irradiance (typically measured in W/m²/nm) and E_{vitD} is the effective solar irradiance for vitamin D synthesis (typically measured in W/m²). The two effects of solar radiation to be balanced in this case are that of vitamin D synthesis (beneficial) and erythema (harmful), using their spectra of action as shown in FIG. 10.

Another preferred embodiment of the system relates to the support for the management of antimicrobial photodynamic treatments of inanimate surfaces (e.g., floors, tissues, personal protective equipment, and others) and the monitoring of related disinfection from viruses and bacteria.

Some examples of application in the case of antimicrobial photodynamic treatment for disinfection of an inanimate surface are given below to further clarify the usefulness of the system of the invention in this case as well. Similar to what has been described above, also in this case the principle of action of the antimicrobial photodynamic treatment is based on the illumination of a surface covered by a layer containing a photo-activated molecule (e.g., protoporphyrin, blue methylene and riboflavin), which, when illuminated, produces singlet oxygen molecules (¹O₂) that attack bacteria or viruses on the surface (oxidative damage), disinfecting the surface progressively and continuously during exposure to sunlight.

For example, one of the possible cases of application is the illumination by solar radiation of a surface (e.g., glass) treated with antimicrobial coating containing riboflavin as a photosensitiser, although the proposed system can be applied to any molecule usable for antimicrobial photodynamic treatment (e.g., methylene blue, protoporphyrin and riboflavin), and to any treated surface (e.g., clothes, gloves, walls and flooring).

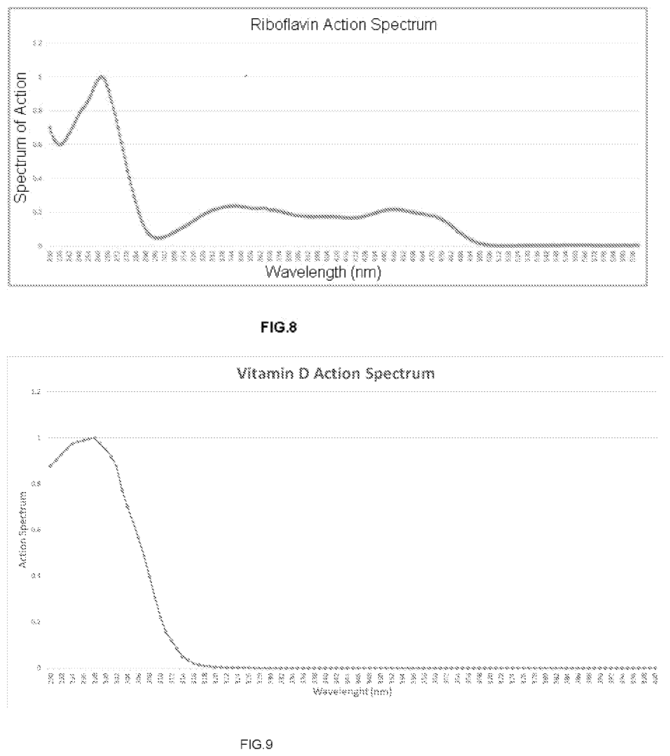

With reference to the example shown in FIG. 8, the considerations made above for the case of application to individuals, apply similarly also in the case of application of the system as a surface treatment support;
The photosensitiser, in this case, is riboflavin, which has the absorption spectrum (and, therefore, the resulting photo-activated antimicrobial action).
for this example, we assume that the effective solar radiation dose (weighed according to the riboflavin spectrum of action) to ensure 99% disinfection from bacteria and viruses (i.e., log reduction of 2.0 log₁₀) is about 4 J/cm², as derived from Eichner et al. (2020) for the specific case of Pseudomonas aeruginosa, one of the most resistant bacteria to this type of treatment (but similar considerations also apply to any other bacterium or virus).

In this second example, we again consider the same three typical non-rainy days in the Oxford region (UK) in April (spring), June (summer) and October (autumn), with the same times considered earlier for the onset of sun exposure (9:00 am, 12:00 pm, and 3:00 pm GMT), as the surface treated with antimicrobial spray or paint containing riboflavin (e.g., glass). The table below considers the spectral solar radiation measured on the three days considered:
the dose of riboflavin-effective radiation accumulated after 2 hours of exposure (for the antimicrobial treatment of an inanimate surface by external sun exposure);
the duration of exposure to solar radiation that would be necessary to disinfect a glass surface, i.e., how long after the onset of sun exposure the threshold of 4 J/cm² of accumulated riboflavin-effective dose is exceeded to have a 99% disinfection.

It should be noted that if the surface in question is worn or, in any case, close to an individual, it is also possible to simultaneously consider the erythema effect of solar radiation on that individual exposed to the sun with or without sun protection (to avoid the risk of sunburn), as already described in detail above.

| | April | June | October |
|---|---|---|---|
| Sun | Riboflavin effective | Riboflavin effective | Riboflavin effective |
| exposure | dose accumulated at | dose accumulated at | dose accumulated at |
| of the | 11:00 am = 4.75 J/cm² | 11:00 am = 15.38 | 11:00 am = 4.78 J/cm² |
| surface | End time of exposure | J/cm² | End time of exposure |
| from 09:00 | for 99% surface | End time of exposure | for 99% surface |
| am | disinfection by | for 99% surface | disinfection by |
| (morning) | photodynamic | disinfection by | photodynamic |
| | antimicrobial | photodynamic | antimicrobial treatment |
| | treatment with | antimicrobial | with riboflavin = 10:45 |
| | riboflavin = 10:45 | treatment with | |
| | | riboflavin = 09:35 | |
| Sun | Riboflavin effective | Riboflavin effective | Riboflavin effective |
| exposure | dose accumulated at | dose accumulated at | dose accumulated at |
| of the | 14:00 = 13.80 J/cm² | 14:00 = 21.35 J/cm² | 14:00 = 9.55 J/cm² |
| surface | End time of exposure | End time of exposure | End time of exposure |
| from 12:00 | for 99% surface | for 99% surface | for 99% surface |
| pm (mid- | disinfection by | disinfection by | disinfection by |
| day) | photodynamic | photodynamic | photodynamic |
| | antimicrobial | antimicrobial | antimicrobial treatment |
| | treatment with | treatment with | with riboflavin = 12:50 |
| | riboflavin = 12:30 | riboflavin = 12:20 | |
| Sun | Riboflavin effective | Riboflavin effective | Riboflavin effective |
| exposure | dose accumulated at | dose accumulated at | dose accumulated at |
| of the | 18:00 = 6.60 J/cm² | 18:00 = 13.96 J/cm² | 18:00 = 2.21 J/cm² |
| surface | End time of exposure | End time of exposure | Exposure end time for |
| from 4:00 | for 99% surface | for 99% surface | 99% surface |
| pm | disinfection by | disinfection by | disinfection by |
| (afternoon) | photodynamic | photodynamic | photodynamic |
| | antimicrobial | antimicrobial | antimicrobial treatment |
| | treatment with | treatment with | with riboflavin = never |
| | riboflavin = 17:00 | riboflavin = 16:25 | reached, because |
| | | | there is not enough |
| | | | solar radiation from |
| | | | 4:00 pm to sunset |

This second application also clearly demonstrates the usefulness of the proposed system to be able to safely monitor the disinfection of inanimate surfaces by means of photodynamic antimicrobial treatment in a reliable and safe manner.

In particular, we can note that the duration of sun exposure required to have a 99% disinfection of a surface (in the example analysed, a glassy surface covered in antimicrobial coating with riboflavin) varies greatly depending on season and time. In particular, we underscore that:
the duration of exposure required for disinfection may be relatively short in some cases, such as mid-day in June when only 20 minutes are needed,
the necessary exposure can instead become much longer in other periods, such as in April, in the afternoon when at least an hour is to be expected (i.e., contact with the infected surface can be risky earlier if you are not aware of this);
in some cases, there is even some possibility of effectively disinfecting the surface (due to lack of sufficient solar radiation), as in October in the afternoon.

The decisions set out above for both phototherapy and inanimate surface treatment cannot be made reliably to date with the means currently available in the state of the art, with the exception of a spectrometer calibrated for real-time monitoring (which, as mentioned, is, however, extremely unlikely that an individual or clinic can have).

The system of the invention for predicting and monitoring spectral solar radiation based on satellite or meteorological data is therefore an advantageous solution for ensuring the effectiveness of treatments based on exposure to solar radiation and/or any phototherapy protocols and reducing the risks of adverse/side effects associated with them. In particular, the effective therapeutic dose is also ensured in the presence of sunscreens and ensuring the simultaneous monitoring of adverse/side effects (in order to avoid them as much as possible) in any geographical location, any weather situation, and any time of day and day of the year (season).

From a structural point of view, in a typical embodiment the system essentially comprises a digital platform to which multiple user groups (e.g., patients, clinical staff) have access.

The digital platform, implemented on cloud servers, includes a data processing unit (including satellite images) and is connected to both a hand-held telecommunications device, which includes a geolocation sensor associated with the data subject (e.g., an individual), and another remote computing device (or hand-held telecommunications device) associated with the data provider (e.g., clinical personnel). This platform can also allow economic-commercial transactions between users, becoming an intermediate multi-party economic platform.

In a typical embodiment thereof, the invention is implemented through a digital system that provides decision support by recommending the solar exposure time of the individual or surface (both in real time and in terms of future planning), according to what is defined by the specific treatment. This time interval is determined (and constantly updated during treatment) based on the dose of effective solar radiation required for each treatment and for each individual or surface, minimising side effects and/or adverse effects, taking into account, for example, the individual's personal skin characteristics (e.g., personal sensitivity to UV radiation) and the spectral filtering properties of both the photosensitising drug (if applied and in what amount) and the solar filter (if applied, or included in the photosensitising drug and in what amount), which affect both the efficacy of treatment and the protection of the individual's skin or surface from side effects/adverse effects.

The digital platform has as its primary interface for the individual (or, in any case, for subject of treatment) an application (app) for portable (mobile) telecommunications devices, while the primary interface for clinical personnel (or, in any case, for the treatment provider(s)) consists of a web portal, which, in any case, can also be accessed from a portable telecommunications device.

The app for the individual provides instructions to correctly deliver the treatment both with and without clinical personnel (who can however supervise it remotely), thanks to the geolocation provided by the GPS signal of the device and the calculation of solar radiation effective for the treatment, and for any other relevant effects, as described above.

The web portal, on the other hand, allows clinical staff to plan treatments adequately, thanks to the prediction of effective solar radiation, as well as to be able to interact directly with the individual remotely and supervise the progress and results of each treatment, even in real time.

Preferably, the course and efficacy of treatment can be further verified remotely by the clinical staff and, possibly, also directly by the individual, thanks to the processing of images of the skin lesions (made with the portable device) through an algorithm for the detection of the severity of the skin lesion, which can also include elements of Artificial Intelligence.

FIG. 7 illustrates an example diagram of a system according to the invention, which supports a treatment requiring the exposure of a user U or a surface S to solar radiation, and comprising a server WS accessible via a telecommunications network WEB, and one or more telecommunications terminals DP arranged in the immediate vicinity of the users U and fitted with geolocation means LOC and a memory MEM for storing specific data D1, for example, personal data of the user U (such as by way of example: phototype, Minimum Erythema Dose—MED, vitamin D concentration in the blood, etc.), or characteristic data of the surface S, relevant for treatment administration.

The illustrated example includes one or more multi-directional communication interfaces Int1 arranged between the server and one or more of the terminals of the users or surfaces subjected to treatment, the interfaces Int1 being configured to send to the server:
geographical location data D2 of the user or the surface,
specific data D1 relevant to the administration of said treatment and,
data D4, if any, relating to sunscreens and/or photosensitising substances for use by the user U and/or antimicrobial spray/paint applied to the surface S relevant for treatment administration (such as: spectral efficacy data (action spectrum), spectral transmittance, spectral absorption, etc.).

The server is also interfaced via one or more additional communication interfaces Int2 with one or more satellite and/or meteorological atmospheric data sources D3, the interfaces Int2 being configured to receive atmospheric data from the sources and send it to said server. Atmospheric data means data from Earth observation satellites or weather forecast data relating to environmental parameters in the geographical position occupied by such users, including also data relating to incident solar radiation. Depending on the application selected, if for user treatments or for surface treatments, one or more sunscreens and/or photosensitising substances and/or antimicrobial sprays/paints associated with one or more users or surfaces subjected to said treatment and relevant to its administration of the treatment are provided.

A processing unit EL is operationally integrated with the server and configured to process operational information INFO for treatment administration and to run a computer program that implements an analysis of the overall therapeutic effect due to atmospheric data D3, to geolocation data D2, specific data D1, and data D4, if any, relating to sunscreens and/or photo-sensitising substances, and/or antimicrobial substances.

The information INFO comprises at least information repeated over time relating to the effective solar radiation dose that the user or the treated surface has received and has yet to receive based on the treatment received.

In the case of application as a support to a user treatment, the system can also provide at least one additional interface Int3 connected to the server WS and configured to make one or more of said operational information processed available to users and/or supervisors. However, in a preferred application example, the system operates with a single connected interface Int1, for example, only the app in use in a single user terminal, for example for self-treatment without the intervention of a supervisor.

In an additional option, the system may also include a wearable device (WR) connected to the portable telecommunication device and provided with sensors to characterise the position, posture and orientation of the individual relative to the position of the sun, so as to further increase the accuracy of solar radiation detection on the areas of the skin of interest of the treatment (e.g., cheek, forehead, etc.), and/or identify the mode of exposure of the individual to the sun, as exposure to full light, shade, partial shade or complete shielding from solar radiation (e.g., inside a house) by comparing a UV sensor and a light meter with solar radiation measured from satellite data.

The invention has been described with reference to a preferred embodiment, but it is intended that equivalent modifications can be made without however departing from the scope of the present industrial property right.

1. A system for supporting a treatment based on the exposure of a user or a surface to solar radiation, the system comprising
a server accessible through a telecommunications network,
one or more telecommunication terminals arranged in the immediate vicinity of the respective users or surfaces and provided with geographic location means and a memory for storing specific data of the user or the surface relevant for the administration of said treatment,
one or more multi-directional communication interfaces between said server and one or more terminals associated with said users or surfaces subjected to said treatment, configured to send to said server geographic user or surface location data, said specific data of the user or surface relevant for the administration of said treatment,
one or more communication interfaces between said server and one or more atmospheric satellite and/or meteorological data sources configured to receive from said sources and send to said server atmospheric satellite and/or meteorological data related to environmental parameters in the geographic location occupied by said users or surfaces, comprising data related to incident solar radiation,
a processing unit operatively integrated with said server and configured to process operational information for the administration of the treatment and to run a computer program which, if run, performs an analysis of the overall therapeutic effect including potentially harmful effects for the user, taking into account said atmospheric satellite and/or meteorological data, said geographic user or surface location data, said specific data of the user or the surface,
said operational information comprising at least repeated information over time relating to an effective or beneficial dose of solar radiation effective for treatment or beneficial for health that the user or surface has received and/or has yet to receive, and for an effective or harmful dose of effective or harmful solar radiation that the user or surface has received and/or has yet to receive.

2. The system according to claim 1, wherein said one or more multi-directional communication interfaces between said server and one or more terminals associated with said users or surfaces subjected to said treatment are further configured to send to said server data relating to supplements or stimulants for the production of systemic substances, such as vitamin D, melatonin, and/or sunscreens and/or systemically and/or topically administrable photosensitising substances for use by the user and/or antimicrobial spray/paint for surfaces relevant for the administration of treatment, and said processing unit being further configured to run said computer program which, if run, performs an analysis taking into account said data relating to supplements or stimulants for the production of systemic substances, such as vitamin D, melatonin, and/or sunscreens and/or systemically and/or topically administrable photosensitising substances for use by the user and/or antimicrobial spray/paint for surfaces that are relevant for treatment administration.

3. The system according to claim 1, wherein said one or more multi-directional communication interfaces comprises an application for portable devices which can be run by means of said terminals.

4. The system according to claim 1, wherein said one or more communication interfaces between said server and one or more atmospheric satellite and/or meteorological data sources comprise a web portal.

5. The system according to claim 1 supporting a treatment based on the exposure of a user or a surface to solar radiation, further comprising at least one further interface operatively connected with said server, configured to provide one or more of said processed operational information to the users and/or subjects supervising the performance of the treatment.

6. The system according to claim 1 supporting a treatment based on the exposure of a user or a surface to solar radiation, wherein said operational information comprises information useful for optimizing desired effects of exposure to solar radiation and minimizing the risks of side effects and adverse effects.

7. The system according to claim 1 supporting a treatment based on the exposure of a user or a surface to solar radiation, wherein said further interface is configured to send said operational information to supervisory subjects arranged at a distance with respect to said users or surface.

8. The system according to claim 9, wherein said solar filters comprise photo protective creams and/or solar screens for use by one or more users.

9. The system according to claim 1 supporting a treatment based on the exposure of a user to solar radiation, wherein said processing unit is configured to run a program for processing operational information for the administration of one or more treatments based on solar radiation, including photodynamic therapy, cosmetic or aesthetic therapy, photo-rejuvenation therapy or photo-retouching of skin photoaged and/or damaged by solar rays, neurofibroma treatment, treatment of local skin reactions, hardening and desensitisation of the skin by solar radiation, treatment of solar polymorphic dermatitis, antimicrobial therapies, heliotherapy, climatotherapy, balneotherapy, treatment by solar radiation of disorders to include actinic keratosis, psoriasis, vitiligo, acne, eczema, tumours and pre-tumours of the skin, DNA damage of the skin, atopic dermatitis, jaundice, lichen planus, circadian disorders, eye disorders, deficiency or maintenance of nutrients essential to human health, with or without the use of products, such as supplements or stimulants for the production of the substance by solar radiation and/or photo protective solar filters, and possibly suggesting the optimal choice between different available options of such products based on said atmospheric data, said geographical location data, and said specific user data based on the processing of the history of such data.

10. The system according to claim 1 supporting a treatment based on the exposure of a user to solar radiation, comprising at least one wearable device for use by a user, connected to a personal user terminal and configured to detect and send to the terminal data related to the orientation/position of the user's body in order to allow the processing unit to process information related to the effective solar radiation on all areas of the user's body and/or identify the individual's mode of exposure to sun, such as exposure in full light, in shade, in partial share or in a situation of complete shielding from the solar radiation.

11. The system according to claim 1 supporting a treatment based on the exposure of a user to solar radiation, wherein said specific data of the user relevant for the administration of said treatment comprise personal data comprising photographs of the individual's skin lesions, and said further interface is configured to send said photographs to a processing and evaluation system in the post-therapy step, preferably through artificial intelligence.

12. The system according to claim 2 supporting a treatment based on the exposure of a surface to solar radiation, wherein said antimicrobial substances associated with one or more surfaces are subjected to said treatment comprising photo-activated substances which, if illuminated by the incident solar radiation, produce oxygen molecules disinfecting the surface.

13. The system according to claim 12, wherein said antimicrobial substances comprise an antimicrobial coating containing a substance selected from riboflavin, methylene blue, protoporphyrin.

14. A method for supporting a treatment based on the exposure of a user or a surface to solar radiation, comprising the following steps
providing a server accessible via web,
providing a plurality of terminals arranged in the immediate vicinity of respective users or surfaces and provided with geographic location means and means for storing specific data of the user or the surface,
providing a first plurality of multi-directional communication interfaces between said server and one or more terminals associated with users or surfaces subjected to said treatment, and sending through said first interfaces to said server geographic location data and specific data of the respective user or surface relevant for the performance of said treatment,
providing a second plurality of communication interfaces between said server and one or more atmospheric data sources and sending through said second interfaces to said server atmospheric data comprising data related to the incident solar radiation in the geographic location occupied by said users or surfaces received from said sources,
providing a processing unit operatively integrated with said server
running through said processing unit a computer program that carries out an analysis of the overall therapeutic effect due to said atmospheric data, said geographical location data, said specific user or surface data,
processing operational information for the administration of the treatment based on the analysis actuated, said information comprising at least repeated information over time related to a dose of effective solar radiation which the user or surface has received and that which must still be received based on the treatment followed.

15. The method according to claim 14, further comprising a step to evaluate the relevance of one or more products, such as supplements or stimulants, sunscreens and/or photosensitising substances and/or antimicrobial sprays/paints associated with one or more users or surfaces subjected to said treatment, and sending to said server said data relating to said products relevant to the performance of the treatment, wherein said processing unit runs through said computer program an analysis of the effect of the overall treatment also due to said data relating to said products and and provides a suggestion as to an optimal choice between different available options of said products based on said atmospheric data, said geographical location data, said specific user data based on the processing of the history of said data.

16. The method according to claim 14, supporting a treatment based on the exposure of a user or a surface to solar radiation, further comprising the step of providing at least one further interface operatively integrated with said processing unit, and providing one or more of said processed operational information to users and/or subjects supervising the performance of the treatment.

17. The system according to claim 1, wherein the effective or beneficial dose of solar radiation effective for treatment or beneficial for health that the user or surface has received and/or has yet to receive relates to one or more of: skin radiation-based phototherapy, ocular radiation-based phototherapy; solar radiation-based phototherapy, vitamin D synthesis; circadian system treatment, and immune system support.

18. The system according to claim 1, wherein the effective or harmful dose of effective or harmful solar radiation that the user or surface has received and/or has yet to receive relates to one or more of erythema; burn; photoageing; and cancer risk.
